(19) United States
(12) Patent Application Publication
(10) Pub. No.: US 2019/0345564 A1
(43) Pub. Date: Nov. 14, 2019

(54) EARLY DIAGNOSIS METHOD AND KIT FOR BREAST CANCER

(71) Applicant: ISTANBUL UNIVERSITESI REKTORLUGU, Istanbul (TR)

(72) Inventors: Oguz OZTURK, Istanbul (TR); Hulya YILMAZ AYDOGAN, Istanbul (TR); Ahmet Ceyhan GOREN, Kocaeli (TR); Allison Pinar ERONAT, Istanbul (TR)

(21) Appl. No.: 16/475,054

(22) PCT Filed: Nov. 13, 2017
(86) PCT No.: PCT/TR2017/050563

§ 371 Date: Jun. 28, 2019

(30) Foreign Application Priority Data
Dec. 29, 2016 (TR) 2016/20070

(51) Int. Cl. C12Q 1/6886

(52) U.S. Cl. CPC C12Q 1/6886; C12Q 2600/118; C12Q 2600/158; C12Q 2600/178

(57) ABSTRACT

The present invention is an in vitro method realized for early diagnosis and prediction of breast cancer, characterized by comprising detection and analysis by means of various mass spectrometer systems for the miRNA (micro RNA) and/or sncRNA, snoRNA derived fragments, tRNA derived fragments in blood or breast tumor tissue.

### TECHNICAL FIELD

The present invention relates to an in vitro method realized for diagnosis of breast cancer. The present invention relates to a method and kit for early diagnosis and prediction of breast cancer by means of detection of non-coded small RNAs (small ncRNA), in general, with size smaller than 400 nucleotide, essentially miRNAs (micro RNA), small endogen interference RNAs (small nucleolus RNAs (snoRNA), snoRNA derived fragments and tRNA derived fragments, etc.) in blood or in the tumor tissue existing in the breast tissue.

### PRIOR ART

Cancer is a substantially mutant disease which is formed by heterozygote genetic and epigenetic changes. The beginning and development of cancer is characterized by various mutations, the deteriorations occurring in chromosomes and the increasing gene expressions. The transcript level, increasing in cancer genomes, is correlated with the inactivation of the tumor suppressing genes and the gene copy number which increases together with the augmentation of the oncogenes.

In case of breast cancers, the responses of the patients to the treatment consist of variable sub-groups because of the different molecular properties of the patients. Therefore, breast cancer is among the cancer types which is the most frequent subject of ‘personal treatment’ researches. Breast cancer is the second most frequent cancer type in humans after lung cancer, and in women, breast cancer is the most frequent cancer type. There are two histological types, namely, invasive and non-invasive. The non-invasive ones are classified as in situ ductal carcinoma and in situ lobular carcinoma, and the invasive ones are classified as invasive ductal carcinoma and invasive lobular carcinoma, tubular carcinoma, invasive cribriform carcinoma, medullar carcinoma, mucinous carcinoma, neuro-endocrine carcinoma, invasive papillary carcinoma, invasive micro-papillary carcinoma, apocrine carcinoma, meta-plastic carcinoma, lipid-rich carcinoma, secretory (juvenile) carcinoma, oncocytic carcinoma and adenoid cystic carcinoma.

According to the data of World Health Organization (2008) and T. R. Ministry of Health (2009), breast cancer incidence is mentioned to be approximately 41 in hundred thousand where every fourth of the women suffering from cancer disease suffers from breast cancer. Various methods are used for diagnosis and these methods have various disadvantages. For the women who are over age forty, the yearly mammography screening decrease mortality by 30-40%, and for the women who are below age 35, the treatment is delimited with manual inspection of the women and doctor evaluation afterwards. Therefore, early diagnosis of the cancer in young patients frequently delays. It has been begun to be discussed in the recent years that the recommended yearly mammography screening for the women over age 40 may have cumulative radiation effect. Breast cancer is a multi-factorial disease which is separated into different groups in terms of clinical, morphological and molecular perspectives. After the detection of breast cancer, the tests like MammaPrint, Oncotype DX and Mammostrat, used frequently in the world in order to predict prognosis, have low cost, however, a more advanced research is required for using frequently in determining the treatment. The most important point is that these platforms depend on biopsy material, in other words, these platforms can only be realized after surgical operation. It is generally used in grading the repeating of the disease and in guiding the treatment process for the patients whose tumor has been removed. The BRCA1/2 and TP53 mutation analyses realized frequently in Turkey are the risk analyses of family breast cancers and they do not provide contribution to the diagnosis or treatment. The usage of routine estrogen (ER), progesterone (PR) receptors and HER2 bio-markers, which support clinical parameters, in the treatment and in prognosis is insufficient. For instance, the target-oriented Trastuzumab treatment used only in HER2 positive patients can give efficient result only in 34% of these patients.

Shortly, in the detection of the disease, distinguishability is needed at a certain level and a diagnosis method is needed which will provide prediction. Said studies include risks in clinical terms and reliability problems.

In the studies made on various cell cultures and on the patient tissues with early-phase breast cancer, it has been mentioned that the expression difference of some proteins and miRNAs related to intracellular signal transmission can be bio-marker in molecular grouping of cancer. In the recent years, in the early detection of cancer cases, proteins and miRNA based markers are also recommended as bio-marker in addition to mammography screening instead of single-gene risk analyses. miRNAs (micro RNA) are small non-coded oligo-nucleotides with single chain having nucleotide length between 18 and 22. miRNAs show their effect by means of leading to changes in expression of various genes by leading to destruction or inhibition of the targeted mRNAs. It has been proven by the studies made before biopsy and after biopsy that these small regulating molecules are resulting from tumor. Again it has been shown that snoRNA (60-300 bp) (it has been described that it is more than 500 in humans) which is smaller than 400 bp and that the endogen small siRNAs (snoRNA derived and tRNA derived fragments) show similar behavior to the miRNAs and that they are effective on various cellular functions.

Today, the detection of the changes in the expression levels of these various small RNAs can be determined by means of micro-array, in other words, by means of hybridization method or Real Time PCR, in other words, in a relative manner. For instance, in case expression is not observed in the micro-array method, it can be considered that hybridization may not be realized. Therefore, the results shall be approved by means of a second method. In this step, some examples selected in random manner are examined by means of the Real Time PCR method and the result is compared with the micro-array findings.

Cancer occurs as a result of accumulation of mutations in various lock molecular pathways. Various different probabilities, formed due to probable changes in the control mechanisms or the realization of the mutations in relation to a pathway or the point where problem is faced on a pathway, lead to some differentiations in the miRNA expression and other small RNA expression variety patterns even among the individuals suffering from the same cancer type.

Because of all of the abovementioned disadvantages, an improvement, related to the diagnosis of breast cancer, is required which includes reliability, time, patient compliancy and cost advantages.

### BRIEF DESCRIPTION OF THE INVENTION

The present invention relates to a novel method and kit providing early diagnosis and prediction of breast cancer in an in vitro manner, for eliminating the above mentioned disadvantages and for bringing new advantages to the related technical field.

Essentially the miRNAs and all of these small non-coded RNAs can be released by the tumor or they can be included in the circulation as a result of tumor necrosis. Due to the small structures and due to convection bonded to specific proteins and lipoproteins, RNA escapes from the activity and it may stay in a non-deteriorated manner. Essentially the miRNAs, all of these small RNA fragments preserve their integrities despite of difficult conditions like heat and pH change, and this shows that they are favorable for routine studies. By Gilad et al, it is put forward that the levels of miRNAs which are nucleic acids (CNA) which circulate in the serum and in the other body liquids due to regulating and cancer-specific activities can be clinically used as bio-marker. Besides various cancer types, the detection of various miRNAs in the blood also in the diabetes patients provides the activities in the wide spectrum.

In the light of the known state of the art, the main object of the present invention is to detect miRNA by means of various mass spectrometer for early diagnosis and prediction of breast cancer particularly in the blood or breast tumor tissue.

Another object of the present invention is to develop a novel method for early diagnosis and prediction of breast cancer, having time, patient compliancy and cost advantages by means of detection of essentially miRNA and/or sncRNA, snoRNA derived fragments, tRNA derived fragments in blood or breast tumor tissue.

Another object of the present invention is to develop a novel method for the early diagnosis and prediction of breast cancer, having time, patient compliancy and cost advantages by means of detection and analysis of miRNA (micro RNA) and/or sncRNA, snoRNA derived fragments, tRNA derived fragments by means of various mass spectrometer in blood or breast tumor tissue.

Another object of the present invention is to develop a kit for early diagnosis and prediction of breast cancer in blood or breast tumor tissue by means of various mass spectrometer systems and which provides detection and analysis of miRNA and/or sncRNA, snoRNA derived fragments, tRNA derived fragments and which is compliant to monitoring after individual diagnosis.

In order to reach said objects, an in vitro method is realized for early diagnosis and prediction of breast cancer.

In order to realize all of the abovementioned objects and the objects which are to be deducted from the detailed description below, the subject matter method comprises detection and analysis by means of various mass spectrometer systems for the miRNA and/or sncRNA, snoRNA derived fragments in blood or breast tumor tissue.

In another preferred application of the invention, said miRNA patterns analyzed for detection and amount comprise at least one or combinations of known miRNAs and/or sncRNA, snoRNA derived fragments, tRNA derived fragments.

In a preferred application of the invention, said miRNA patterns analyzed for detection and amount comprise detection and analysis of amount of the decrease in the expression level of at least one or combinations of known miRNAs and/or sncRNA, snoRNA derived fragments, tRNA derived fragments.

In another preferred application of the invention, said miRNA patterns analyzed for detection and amount comprise detection and analysis of amount of the increase in the expression level of at least one or combinations of known miRNAs and/or sncRNAs, snoRNA derived fragments, tRNA derived fragments.

In another preferred application of the invention, said breast cancer is at least one selected from in situ ductal carcinoma and in situ lobular carcinoma, invasive ductal carcinoma and invasive lobular carcinoma, tubular carcinoma, invasive cribriform carcinoma, medullar carcinoma, mucinous carcinoma, neuro-endocrine carcinoma, invasive papillary carcinoma, invasive micro-papillary carcinoma, apocrine carcinoma, meta-plastic carcinoma, lipid-rich carcinoma, secretory (juvenile) carcinoma, oncocytic carcinoma, adenoid cystic carcinoma.

In another preferred application of the invention, the following steps are provided:
a) providing blood or breast tumor tissue sample isolated from at least one individual,
b) detecting the expression level of miRNA and/or sncRNA, snoRNA derived fragments, tRNA derived fragments within this sample,
c) interpreting this level in statistical terms.

In another preferred application of the invention, the following steps are provided:
a) providing blood or breast tumor tissue sample isolated from at least one individual,
b) detecting the increase in the expression level and analysis of the amount of at least one of miRNA and/or sncRNA, snoRNA derived fragments, tRNA derived fragments selected from the known miRNAs within this sample,
c) interpreting this level in statistical terms.

In another preferred application of the invention, the following steps are provided:
a) providing blood or breast tumor tissue sample isolated from at least one individual,
b) detecting the decrease in the expression level and analysis of the amount of at least one of miRNA and/or sncRNA, snoRNA derived fragments, tRNA derived fragments selected from the known miRNAs within this sample,
c) interpreting this level in statistical terms.

In another preferred application of the invention, in order to provide detection and/or analysis of miRNA and/or sncRNA, snoRNA derived fragments and tRNA derived fragments, a kit is provided which is used in an individual manner in early diagnosis and prediction of breast cancer.

In another preferred application of the invention, in order to provide detection and/or analysis of miRNA and/or sncRNA, snoRNA derived fragments and tRNA derived fragments, a kit is provided which is used in an individual manner in early diagnosis and prediction of breast cancer by means of various mass spectrometer systems.

Said kit provides realization of the detection and analysis of the decrease in the expression level of at least one of known miRNA and/or sncRNA, snoRNA derived fragments and tRNA derived fragments.

Said kit provides realization of the detection and analysis of the increase in the expression level of at least one of known miRNA and/or sncRNA, snoRNA derived fragments and tRNA derived fragments.

### DETAILED DESCRIPTION OF THE INVENTION

In this detailed description, the subject matter diagnosis method is explained with references to examples without forming any restrictive effect only in order to make the subject more understandable.

In said diagnosis method, the early diagnosis and prediction of breast cancer in an in vitro manner are provided by means of various mass spectrometry systems developed.

### EXAMPLE

Within the scope of the present invention, the fingerprints which are unique for the sub-species of breast cancer are determined by means of miRNAs and/or sncRNA, snoRNA derived fragments, tRNA derived fragments detected in our studies and selected by searching literature, and analysis algorithm is formed. By using samples which are worked on simultaneously with the microarray and various cell spectrometer systems, a novel method is developed by means of various mass spectrometer systems which are one of the most precise methods of today, and this method is standardized and the kit, comprising miRNA and/or sncRNA, snoRNA derived fragments, tRNA derived fragments, is formed. By using these kits; only by means of blood analysis, it is targeted to early-detect cancer, the tumors of early-phase and late-phase or metastasis making tumors and even the tumors with very small dimensions. Fingerprint which is unique for sub-species of breast cancer is formed, and by using the blood or tumor sample of the patient, the diagnosis of the cancer type is realized and the prognosis is determined. At the same time, the effect of breast tumor mass diameter on the amount of peripheral miRNAs and/or sncRNA, snoRNA derived fragments, tRNA derived fragments is compared and the correlation in between the researched. The kit, which we designed and which is formed by miRNA and/or sncRNA, snoRNA derived fragments, tRNA derived fragments, is unique and compliant to various mass spectrometer systems and can be used for diagnosis and monitoring of breast cancer only from blood samples.

All miRNAs, known from our previous studies, are scanned in blood, serum and cell culture materials and an algorithm is formed. The validation of microarray study has been realized by comparing 10 miRNA expressions, which are randomly selected by means of the Real-Time PCR method, with the microarray result.

The traces, which is unique to the sub-species of breast cancer and collected from the array studies realized by means of cell strains and sample standardization, have been determined by making method validations, measurement uncertainty calculations, and the kit has been developed for use in routine measurements.

In routine controls of breast cancer for the women who are below age 35, the treatment is delimited with manual inspection of the women and therefore, the early diagnosis of the cancer in young patients frequently delays. It has been begun to be discussed in the recent years that the recommended yearly mammography screening for the women over age 40 may have cumulative radiation effect. The kit, formed thanks to the present invention, can be used for yearly monitoring millions of young women between age 15 and 40 which are frequently manually inspected and for the yearly monitoring of women over age 40 where routine radiological monitoring is recommended. This kit is for diagnosis and monitoring of cancer in a very different manner from the kits which determine DNA-based risk, and it does not have side effect and it only works by taking blood and/or tissue sample. By means of a unique operation design, the fingerprints of miRNA and/or sncRNA, snoRNA derived fragments, tRNA derived fragments of breast cancer patients are obtained and a kit is formed which is based on various mass spectrometer systems and which is rapid and low-cost. When compared with the present methods, the most prominent superiority of the present invention is that real measurement can be made by means of mass measurement method.

The aim of this kit is to diagnose cancer in the early age and in the early-phase and to diagnose the masses which are difficult to monitor in the mammography screening, and moreover to provide planning of the treatment. Moreover, algorithms can be created for the cases where resistance is shown to chemotherapy drugs. For example, some of the miRNAs which can be scanned are as follows: miR-let-7i, miR-100, miR-107, miR-10a, miR-10b, miR-125b, miR-128,miR-130a, miR-132, miR-140-5p, miR-141, miR-148a, miR-152, miR-155, miR-15a, miR-15b, miR-16, miR-17, miR-182, miR-186, miR-193b, miR-199b-3p, miR-200b, miR-200c, miR-204, miR-205, miR-206, miR-21, miR-210, miR-212, miR-22, miR-222, miR-25, miR-27a, miR-27b, miR-29a, miR-31, miR-328, miR-429, miR-485-5p,miR-489, miR-495, miR-93, miR-96, etc. These miRNAs can be observed to be increased/decreased in different types of breast cancers in different combinations and even for different persons.

As a result, in a surprising manner, a novel method is developed which provides detection/prediction of cancer and early diagnosis in patients suffering from breast cancer in the in vitro medium. An important marker of the increase/decrease in the expression level of at least one of the selected miRNAs and/or sncRNA, snoRNA derived fragments, tRNA derived fragments has been evaluated for early detection/prediction of cancer in the patients suffering from breast cancer.

Each of or combinations of the increase/decrease in the expression level of the miRNAs and/or sncRNA, snoRNA derived fragments, tRNA derived fragments selected from the miRNAs known according to the invention may be a pre-indicator in the prediction of repeating probability of breast cancer.

By means of the measurements and tools of the subject matter method described above, the subject matter kit can be used individually in early diagnosis of the prostate cancer, where said kit provides detection and/or change level analysis of miRNA and/or sncRNA, snoRNA derived fragments, tRNA derived fragments based on these and where at least one or all of the method steps are used.

1. An in vitro method is realized for early diagnosis and prediction of breast cancer, characterized by comprising detection and analysis by means of various mass spectrometer systems for the miRNA and/or sncRNA, snoRNA derived fragments in blood or breast tumor tissue.

2. A method according to claim 1, wherein said miRNA patterns analyzed for detection and amount comprise detection and analysis of amount of the increase in the expression level of at least one or combinations of known miRNA and/or sncRNA, snoRNA derived fragments, tRNA derived fragments.

3. A method according to claim 1, wherein said miRNA analyzed for detection and amount comprise detection and analysis of amount of the decrease in the expression level of at least one or combinations of known miRNA and/or sncRNA, snoRNA derived fragments, tRNA derived fragments.

4. A method according to claim 1, wherein said breast cancer is at least one selected from in situ ductal carcinoma and in situ lobular carcinoma, invasive ductal carcinoma and invasive lobular carcinoma, tubular carcinoma, invasive cribriform carcinoma, medullar carcinoma, mucinous carcinoma, neuro-endocrine carcinoma, invasive papillary carcinoma, invasive micro-papillary carcinoma, apocrine carcinoma, meta-plastic carcinoma, lipid-rich carcinoma, secretory (juvenile) carcinoma, oncocytic carcinoma, adenoid cystic carcinoma.

5. A method according to claim 1,
wherein the following steps are provided:
a) providing blood or breast tumor tissue sample isolated from at least one individual,
b) detecting the expression level of miRNA and/or sncRNA, snoRNA derived fragments, tRNA derived fragments within this sample,
c) interpreting this level in statistical terms.

6. A method according to claim 1, wherein the following steps are provided:
a) providing blood or breast tumor tissue sample isolated from at least one individual,
b) detecting the increase in the expression level and analysis of the amount of at least one of miRNA and/or sncRNA, snoRNA derived fragments, tRNA derived fragments selected from the known miRNAs within this sample,
c) interpreting this level in statistical terms.

7. A method according to claim 1,
wherein the following steps are provided:
a) providing blood or breast tumor tissue sample isolated from at least one individual,
b) detecting the decrease in the expression level and analysis of the amount of at least one of miRNA and/or sncRNA, snoRNA derived fragments, tRNA derived fragments selected from the known miRNAs within this sample,
c) interpreting this level in statistical terms.

8. A kit for early diagnosis and prediction of breast cancer according to claim 1, wherein in order to provide detection of miRNA and analysis of the change level of miRNA, it comprises mass spectrometer systems.

9. A kit for early diagnosis and prediction of breast cancer according to claim 1, wherein in order to provide detection and/or analysis of the increase in expression level of miRNA and/or sncRNA, snoRNA derived fragments and tRNA derived fragments, it comprises mass spectrometer systems.

10. A kit for early diagnosis and prediction of breast cancer according to claim 1, wherein in order to provide detection and/or analysis of the decrease in expression level of miRNA and/or sncRNA, snoRNA derived fragments and tRNA derived fragments, it comprises mass spectrometer systems.
